(19) United States
(12) Patent Application Publication
(10) Pub. No.: US 2009/0130747 A1
(43) Pub. Date: May 21, 2009

(54) System and Method of Enhancing Production of Algae

(75) Inventors: Wu Wen-Teng, Tainan City (TW); Chih-Hung Hsieh, Tainan City (TW)

Correspondence Address: WPAT, PC;INTELLECTUAL PROPERTY ATTORNEYS, 2030 MAIN STREET, SUITE 1300, IRVINE, CA, 92614, US

(73) Assignees: Mon-Han Wu, Tainan City (TW); SUSTINEO BIOTECHNOLOGY CO., LTD, Tainan City (TW)

(21) Appl. No.: 11/941,568

(22) Filed: Nov. 16, 2007

(51) Int. Cl. C12M 1/42; G01N 21/01

(52) U.S. Cl. 435/292.1; 250/432 R

(57) ABSTRACT

The present invention relates to a system and method of enhancing production algae, comprising a container containing liquid, algae and plates made from a kind of material for absorbing and scattering light. The shape, dimension and arrangement of the plates are optimized.

### FIELD OF THE INVENTION

The present invention relates to a system and method of enhancing production of algae.

### DESCRIPTION OF PRIOR ART

Algae are applicable to green fuel generation such as bio-diesel, hydrogen, alcohol, methanol, and even itself; to environmental protection such as absorption of carbon dioxide and purification of waste; and to nutrient generation such as health food, manure, feed, and fishery resources. Due to the economic value, techniques for these applications and mass production become important issues for years.

Lots of applications have been realized. For instance, GB2254858 discloses a method of power generation characterized in that the fuel comprises algae so that obtaining the fuel from algae ensures an inexhaustible supply of easily obtainable fuel; U.S. Pat. No. 7,135,308 discloses a process of producing ethanol from starch-accumulating filament-forming or colony-forming algae as well as process of producing bio-diesel from the biomass remaining after ethanol production; WO2007010068 discloses a composition comprising algae, which is intended for the treatment of industrial and urban solid and semi-solid (sludge) waste containing biodegradable organic material and which is applied to said waste; JP2006320320 discloses a method for producing a high-temperature extract composition of marine alga for producing foods including seasoning, a cosmetic and a health food.

The technique for mass production of algae, however, is limited since the intensity of incoming light would be weaken by the concentrated algae itself, the average utility rate of light per unit weight of algae, based on Lambert-Beer's Law, is thus restricted.

To reduce the effect, the container designed for culture of algae traditionally must be thin enough or in the form of pipe. Both Zukunftsagentur Brandenburg GmbH and Fraunhofer IGB, for example, announced photo-bioreactors with a few centimeters thick to provide algae cultures of great density with sufficient light, yet the space utility becomes another bottleneck.

US20030113081 discloses that a plate incorporating a number of optical fiber threads is designed for intercepting the incoming light from one surface and conducting it to another one and is supposedly suitable for algae growth. It's costly obviously and it is without showing a method for applying breeding algae in an effective way, yet. So it is still difficult to culture algae in a large scale by current achievements.

### SUMMARY OF THE INVENTION

A system and a method of enhancing production of algae by arranging plates made from transparent or semi-transparent materials which can scatter or redirect the incoming light. The shape, dimension and arrangement of the plates are optimized according to the path and extinction coefficient of the chosen material to enhance light density in the system.

### BRIEF DESCRIPTION OF THE DRAWINGS

FIG. 1 shows a reactor comprising an open tank in order to mimic traditional culture pool which outer length, width, and height was 49 cm, 36 cm, and 25 cm, respectively. Four hollow, transparent acrylic-sheets which length, width, and height is 25 cm, 5 cm, and 21 cm, respectively were installed and parallel to each other with the distance of 5 cm (a) vertical view (b) lateral view.

FIG. 2 shows the change chart of F_{ab} value which changes based on the biomass concentration.

FIG. 3 shows the increase of biomass as the time increases.

### DETAILED DESCRIPTION OF THE INVENTION

During the process of algae culturing, the concentration of algae would increase and the intensity of incoming light for photosynthesis per unit volume of algae would thus decrease as the time goes by. Such a condition is clearly the limiting term for algae growth. Based on Lambert-Beer's Law, the condition can be inferred and modeled mathematically as follows:

Lambert-Beer's Law:

I(z)=I₀·e^{-(C}^{_{b}}^{·K}^{_{b}}^{+K}^{_{w}}^{)·Z}

I(z): light intensity of location
I₀: light intensity at the surface
C_{b}: biomass concentration
z: length of light path
K_{b}: Extinction coefficient for biomass
K_{w}: Extinction coefficient for water

The average light intensity Iₐᵥ can thus be expressed as

$${I}_{av} = \frac{{\int }_{0}^{L} {e}^{- ( {C}_{b} \cdot {K}_{b} + {K}_{w} ) \cdot Z} d z}{{\int }_{0}^{L} d z} .$$

Photon flux absorbed by the biomass unit, F_{ab} (μEg⁻¹s⁻¹), is defined as

F_{ab}=Iₐᵥ·K_{b},

and this value can be estimated by measuring the difference of light intensity between incoming and outgoing light.

The inventions include a system comprising a container containing liquid, algae in the container, and a number of plates made from a kind of material for absorbing and scattering light, which is positioned in the container with optimized shape, dimension, and arrangement according to the material and the source of light; and a method of enhancing light density in a container, comprising a design of plates which comprise materials for absorbing and scattering light with corresponding shape and dimension; and an arrangement of plates, which maximizes the light density in the container.

The container can be a tank or pond, the liquid is marine water with Walne's medium or other mediums for the corresponding algae, the algae for mass production, such as Nannochloris atomus Butcher, Nannochloris maculata Butcher; Nannochloropsis gaditana Lubian, Nannochloropsis oculata, Nannochloropsis salina, Tetraselmis chuii, Chaetoceros gracilis, Rhodomonas salina, Isochrysis galbana, Prorocentrum micans, Pavlova lutheri, Skeletonema costatum, Phaeodactylum tricornutum, Tetraselmis chuii, or Thalassiosira pseudonana, are chosen according to the purpose, and the plates is made from acrylic-sheet, mirror, prism, plastic bag, transparent fluid wrapped in transparent container, or other transparent or semi-transparent materials. The plates can scatter or redirect the incoming light from one surface and forward it to the other surfaces so that it can improve the light density in the deeper part of the container, especially under in the condition that the concentration of the breeding algae is too concentrated to transparent. The shape, the dimension and the arrangement of the plates can be optimized according to the path of light and extinction coefficient of the chosen material. The effect can be evaluated by measuring F_{ab} mentioned above.

The plates can be integrated into existing bioreactors for algae culturing and its expense can be economic.

### EXAMPLE

The examples below are non-limiting and are merely representative of various aspects and features of the present invention.

The following example demonstrates the achievement of increasing the biomass of algae via the invention.

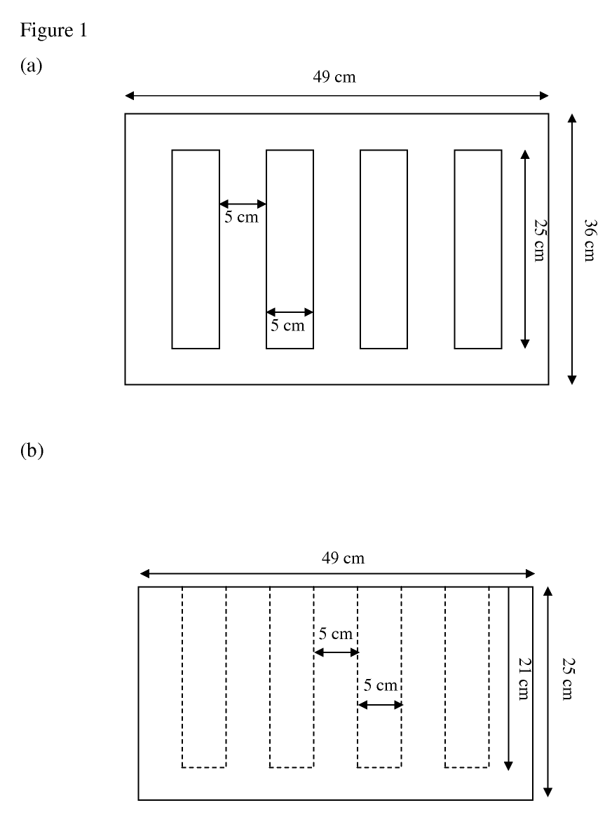

The alga for test was Nannochloropsis oculata; the medium for culture was Walne's medium with artificial marine water and the total volume was 18 L; the reactor was an open tank in order to mimic traditional culture pool which outer length, width, and height was 49 cm, 36 cm, and 25 cm, respectively and inner length, width, and height was 43 cm, 31.5 cm, and 24 cm, respectively, and which surroundings were made from opaque plastics (FIG. 1). The light source consisted of four 500 W Halogen lamp and irradiates the reactor from up to down with the distance of 20 cm to the surface of medium. Four hollow, transparent acrylic-sheets which length, width, and height is 25 cm, 5 cm, and 21 cm, respectively were installed and parallel to each other with the distance of 5 cm (FIG. 1).

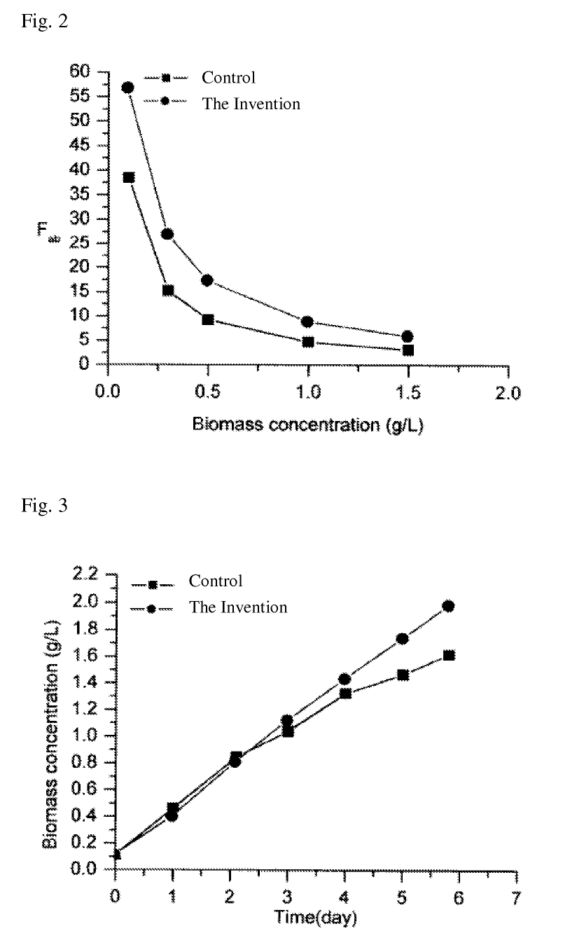

As shown in FIGS. 2 and 3, it provided that the F_{ab} in the experiment was higher than control; and the increase in biomass was about 18.4%.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

What is claimed is:

1. A system of enhancing production of algae comprising a container containing liquid; algae in the container; and plates made from a kind of material for absorbing and scattering light, which is positioned in the container with optimized shape, dimension, and arrangement according to the material and the source of light.

2. The system of claim 1, wherein the container is a tank or pond.

3. The system of claim 1, wherein the liquid is marine water with Walne's medium or other mediums for the corresponding algae.

4. The system of claim 1, wherein the algae is Nannochloris atomus Butcher, Nannochloris maculata Butcher, Nannochloropsis gaditana Lubian, Nannochloropsis oculata, Nannochloropsis salina, Tetraselmis chuii, Chaetoceros gracilis, Rhodomonas salina, Isochrysis galbana, Prorocentrum micans, Pavlova lutheri, Skeletonema costatum, Phaeodactylum tricornutum, Tetraselmis chuii, or Thalassiosira pseudonana.

5. The system of claim 1, wherein the material for scattering or redirecting incoming light is acrylic-sheet, mirror, prism, plastic bag, transparent fluid wrapped in transparent container, or other transparent or semi-transparent materials.

6. The system of claim 5, wherein the material is in shape of plate or others optimized according to the path of light and extinction coefficient of the chosen material and the source of light.

7. The system of claim 6, which comprises at least two plates.

8. The system of claim 7, wherein the two plates are positioned according to the path of light and extinction coefficient of the chosen material and the source of light.

9. A method of enhancing light density in a container, comprising a design of plates which comprise materials for absorbing and scattering light with corresponding shape and dimension; and an arrangement of plates, which maximizes the light density in the container.

10. The method of claim 9, wherein the material for absorbing and scattering light is acrylic-sheet, mirror, prism, transparent fluid wrapped in transparent container, or other transparent or semi-transparent materials.

11. The method of claim 9, wherein the shape and dimension of the plates is designed according to the path of light and extinction coefficient of the chosen material and the source of light.

12. The method of claim 9, wherein the plates are arranged according to the path of light and extinction coefficient of the material.

13. The method of claim 9, the effects is evaluated by measuring photon flux absorbed by the biomass unit, F_{ab}.
